(19) United States
(12) Patent Application Publication
(10) Pub. No.: US 2009/0157163 A1
(43) Pub. Date: Jun. 18, 2009

(54) Retrieval device

(76) Inventors: Eric Cheng, New Richmond, OH (US); Jeffrey C. Smith, Cloverdale, IN (US); James Teague, Spencer, IN (US); Mohamed Fazni Aziz, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/314,752

(22) Filed: Dec. 16, 2008

Related U.S. Application Data
(60) Provisional application No. 61/006,053, filed on Dec. 17, 2007.

(51) Int. Cl. A61F 2/06; A61B 19/00

(52) U.S. Cl. 623/1.11; 128/898

(57) ABSTRACT

A retrieval device is disclosed. The retrieval device may include a sheath including a lumen extending longitudinally through the sheath, an elongate member slidably disposed within the lumen, and a basket at a distal end of the elongate member. The basket may include a plurality of legs formed integrally with the elongate member. The elongate member and the basket may be formed of a single wire, and the legs may be made by splitting a length of the single wire.

### CROSS REFERENCE TO RELATED APPLICATIONS

This application is based upon and claims the benefit of priority under 35 U.S.C. §119(e) of U.S. Provisional Application No. 61/006,053 to Cheng et al. filed on Dec. 17, 2007.

### FIELD OF THE INVENTION

This disclosure relates generally to medical devices and, more particularly, to retrieval devices having integrally formed wire baskets, and methods of manufacturing and using the same.

### BACKGROUND OF THE INVENTION

Extractors have been used for the removal of stones, calculi, and other foreign matter from within the body. One type of extractor has an end effector configured to form a basket at the distal end of a drive wire which extends into the body. The drive wire extends within a sheath and connects to an actuation handle located external to the body. The basket may be collapsed within the sheath to achieve a reduced diameter profile, and may be opened when it extends beyond the sheath. Once opened, a targeted stone may be captured within the basket. The captured stone, along with the extractor, may then be removed from the body. The baskets of such extractors may typically be formed by soldering, welding, or otherwise mechanically joining wires to form tips at distal and proximal ends of the basket. The drive wire may also be soldered, welded, or mechanically connected (mechanical joint) to the proximal end of the basket to couple the basket to the handle which actuates the basket.

In typical baskets, the mechanical joints that are used to form the tips may hinder access to the targeted stone and may increase the possibility of tissue damage within the body. In addition, the mechanical joints may also obstruct the sliding of the drive wire within the sheath during actuation. The size of the basket and the mechanical joint may also necessitate the use of a larger diameter sheath (to pass the drive wire through), increasing patient discomfort or hindering the ability of the device to be passed through a scope, such as a ureteroscope.

The present disclosure provides retrieval devices and methods of manufacturing and using the same that avoid some or all of the aforementioned shortcomings of existing devices.

### SUMMARY OF THE INVENTION

In an exemplary embodiment of the present disclosure, a retrieval device is disclosed. The retrieval device may include a sheath including a lumen extending longitudinally through the sheath, an elongate member slidably disposed within the lumen, and a basket at a distal end of the elongate member. The basket may include a plurality of legs formed integrally with the elongate member. The elongate member and the basket may be formed of a single wire and the legs may be made by splitting a length of the single wire.

Various embodiments of the retrieval device may include one or more of the following aspects: a tip at a distal end of the plurality of legs, the tip may be formed integrally with the elongate member and the plurality of legs; the elongate member, the basket and the tip may be formed integrally without joints; the plurality of legs may be formed by one or more cuts extending all the way through a diameter of the elongate member; one cut may form two legs of the plurality of legs and two cuts may form four legs of the plurality of legs; the retrieval device may further include a handle at a proximal end of the elongate member, the handle may be configured to transition the basket from a retracted configuration to an expanded configuration; the retracted configuration may be a configuration where the basket is disposed within the lumen and the expanded configuration may be a configuration where the basket is disposed external to the lumen; the basket in the expanded configuration may form a closed cage with the plurality of legs having a bulbous shape; the basket in the expanded configuration may form an open cage with the plurality of legs forming prongs of a grasping device; the elongate region and the plurality of legs may be made of nitinol; and a diameter of the elongate region may be between about 8 mils and about 14 mils.

Another embodiment of the invention may include a method of manufacturing a medical device. The method may include splitting a length of wire at a distal end of the wire to form a plurality of legs separated by longitudinal slots, and shaping the plurality of legs to define a basket. The method may further include disposing the wire within a longitudinal lumen of a sheath such that the basket is proximate to a first end of the sheath.

Various embodiments of the method may include one or more of the following aspects: splitting the length of wire may include one of laser cutting, chemical etching, die cutting, Electronic Discharge Machining (EDM), and mechanically slicing; splitting a length of wire may include cutting all the way through a diameter of a section of the wire to form two legs of the plurality of legs; splitting a length of wire may further include rotating the wire and cutting the section of the wire all the way through the diameter to form four legs of the plurality of legs; the wire may be made of nitinol; the diameter of the wire may be between about 8 mils and about 14 mils; the length of wire may be separated from a distal-most end of the wire by a small section of the wire; shaping the plurality of legs may include curving the plurality of legs to form a closed basket having a bulbous shape; the length of wire may abut a distal-most end of the wire; shaping the plurality of legs may include curving the plurality of legs to form an open cage with the plurality of legs forming prongs of a grasping device; disposing the wire may include slidably disposing the wire such that the basket is configured to transition from a first configuration to a second configuration, the first configuration may be a configuration where the basket is disposed within the lumen and the second configuration may be a configuration where the basket is disposed external to the lumen.

Yet another embodiment of the invention may include a method of removing matter from the body of a patient. The method may include providing a medical device which includes a sheath defining a lumen, an elongate member slidably disposed within the lumen, and a basket having a plurality of legs. The elongate member and the basket may be made of a single wire, and the plurality of legs may be formed by splitting a length of the single wire at a distal end of the single wire. The method may also include advancing the medical device to a treatment site within the body of the patient, capturing the matter within the basket of the medical device, and removing the medical device from the body of the patient.

Various embodiments of the method may include one or more of the following aspects: the elongate member and the basket may be formed integrally without joints; capturing the matter may include transitioning the basket from a retracted configuration to an expanded configuration, the retracted configuration may be a configuration where the basket is disposed within the lumen and the expanded configuration may be a configuration where the basket is disposed external to the lumen; the length of the single wire may be separated from a distal-most end of the single wire by a small section of the single wire, and the basket may be configured to have a bulbous shape in the expanded configuration; the length of the single wire may abut a distal-most end of the single wire, and the basket may be configured to form an open cage with the plurality of legs forming prongs of a grasping device in the expanded configuration; the method may further include immobilizing the matter with the plurality of legs of the basket; the method may further include fragmenting the matter; the method may further include sweeping at least a portion of the matter from the treatment site with the plurality of legs of the basket; the matter may be one of a kidney stone, a struvite, an uric acid stone, a cystine stone, and a solid deposit; one cut may forms two legs of the plurality of legs; and two cuts may form four legs of the plurality of legs.

It is to be understood that both the foregoing general description and the following detailed description are exemplary and explanatory only, and are not restrictive of the invention as claimed.

### BRIEF DESCRIPTION OF THE DRAWINGS

FIG. 1 is a schematic illustrating a cross-sectional view of a retrieval device in a retracted configuration, according to an exemplary embodiment of the present disclosure.

FIG. 2 illustrates the retrieval device FIG. 1 in an extended configuration.

FIG. 3 is a schematic illustration of a laser cutting process used to form the basket of the retrieval device of FIG. 1.

FIG. 4 is a schematic illustration of the basket of the retrieval device of FIG. 1.

FIG. 5 is a schematic illustration of another embodiment of a basket of a retrieval device.

### DETAILED DESCRIPTION

Exemplary embodiments of the present disclosure are illustrated in the accompanying drawings. Wherever possible, the same reference numbers will be used throughout the drawings to refer to the same or like parts.

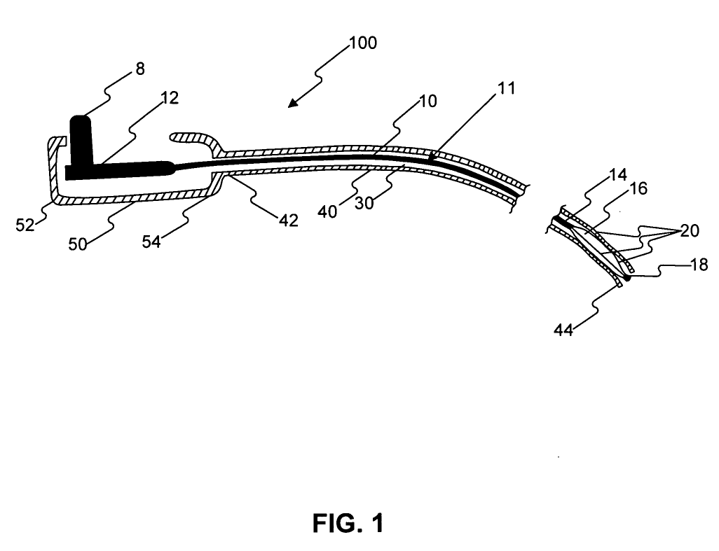

FIG. 1 illustrates a retrieval device 100 according to an exemplary embodiment of the present disclosure. The device 100 includes a sheath 40 with a lumen 30 extending longitudinally from a sheath proximal end 42 to a sheath distal end 44. A handle 50 with a handle proximal end 52 and a handle distal end 54 may be attached to the sheath proximal end 42. An inner member 11 which includes an elongate region 10, a basket 16, and a tip 18 may be slidably disposed within the lumen 30. The elongate region 10 of the inner member 11 may include a member proximal end 12 and a member distal end 14, and may be disposed within the lumen 30 such that the member proximal end 12 may be located within the handle 50 and the member distal end 14 may be disposed within the sheath distal end 44.

The basket 16 may be attached to the member distal end 14. The basket 16 may be comprised of a plurality of legs 20. The plurality of legs 20 may include any number of legs (2, 3, 4, 5, etc.). The proximal end of these plurality of legs 20 may join together and be attached to the member distal end 14 and the distal end of the plurality of legs 20 may join together and be attached to the tip 18. In some embodiments, the tip 18 may possess an atraumatic configuration. In these embodiments, the tip 18 may possess any atraumatic configuration known in the art. For example, the tip 18 may be rounded, crimped, and/or capped.

The legs 20 may be attached to the elongate region 10 (at the member distal end 14) and the tip 18 in any conventional manner (such as, welding, soldering, gluing, crimping, or other mechanical connections). However, as will be described in connection with certain preferred embodiments, the legs 20 may be integrally formed with the elongate region 10 and/or the tip 18. That is, the legs 20 may be formed out of the same piece of material as the elongate region 10, such that the legs 20 and the elongate region 10 have a continuous material formation (for example, no welding, soldering, gluing, mechanical connections, etc. to connect two distinct parts together). In some embodiments, the tip 18 may also be integrally formed with the legs 20 and the elongate region 10. In these embodiments, the legs 20 may be created such that one end of the plurality of legs 20 is integral with the elongate region, and the other end is integral with the tip 18. In some other embodiments, only the legs 20 and the elongate region 10 may be integrally formed, while the legs 20 may be joined at the tip 18 in a conventional manner. It is also contemplated that, in some embodiments, the tip 18 may be eliminated and the legs 20 may extend all the way to the distal-most end of the inner member 11.

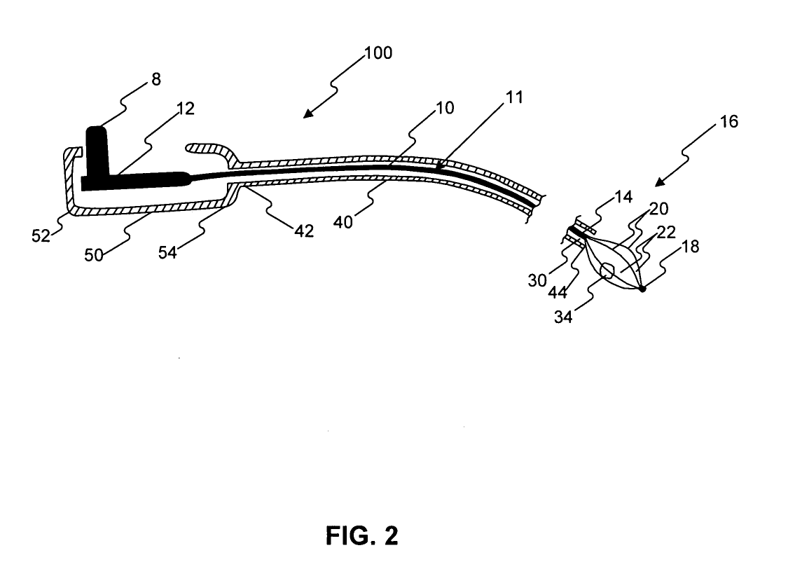

In the retracted configuration, illustrated in FIG. 1, the basket 16 may be disposed within the lumen 30. Sliding the inner member 11 within the sheath 40 may transition the basket 16 (slide the basket 16 out of the lumen 30 at the sheath distal end 44) to an expanded configuration as illustrated in FIG. 2. The basket 16 may be transitioned to the expanded configuration to capture and retrieve stones 34 or other calculi within the body.

The handle 50 of the device 100 may be configured to assist in transitioning the basket 16 between the retracted configuration (FIG. 1) and the expanded configuration (FIG. 2). The handle 50 may be configured to induce relative displacement between the sheath 40 and the inner member 11 to slide the basket 16 into and out of the lumen 30. In an exemplary embodiment, the handle 50 may include a thumb slide 8 (or other conventional mechanisms) attached to the member proximal end 12. Sliding the thumb slide 8 from a handle proximal end 52 to a handle distal end 54 may move the inner member 11 in a distal direction relative to the handle 50, and thus, transition the basket 16 from the retracted configuration to the expanded configuration. In this embodiment, sliding the thumb slide 8 back to the handle proximal end 52 may move the inner member 11 towards the handle 50, thereby transitioning the basket 16 back to the retracted configuration. In another embodiment, the inner member 11 may remain stationary with respect to the handle 50, and the thumb slide 8 may be configured to move the sheath 40 to transition the basket 16 between the expanded and retracted configurations. It is also contemplated that the handle 50 may assist the transitioning of the basket 16 by other means.

In certain preferred embodiments of the invention, including those described herein, the inner member 11 may be a single solid wire integrally formed with the basket 16 and the tip 18, the wire having a relatively small and uniform diameter along its length. The inner member 11, however, may also be formed from a flexible rod, tube, hypotube, cannula, stent, or other piece of biocompatible material or combination of biocompatible materials known in the art. Such materials may include, but are not limited to, nylon, urethane, polyamide, PEBAX, stainless steel (such as 300 and 400 series including 316L, 304, 445), cobalt, chromium, nickel, titanium, nitinol, thermoforming plastic, polytetrafluoroethylene (“PTFE”), and expanded polytetrafluoroethylene (“ePTFE”). The inner member 11 may also be a metal coated with a polymer and may have one or more layers of material.

The elongate region 10 may be solid or hollow, and may be substantially cylindrical. In some embodiments, the diameter of the elongate region 10 may vary along the length. For instance, the diameter of the elongate region 10 may be greater near the member proximal end 12 than the member distal end 16. In these embodiments, the varying diameter of the elongate region 10 may make the inner member 11 stiffer near the handle 50 than near the basket 16. The varying stiffness of the inner member 11 may assist in sliding the inner member 11 in response to sliding of the thumb slide 8, and/or inserting the device 100 through tortuous anatomy of a patient. Alternatively, the elongate region 10 may have a substantially uniform cross-section along its length. In an exemplary embodiment, the elongate member 10 may be formed from a nitinol wire.

The overall length and diameter of the inner member 11 and/or the elongate region 10 may vary depending on the application. For example, a relatively long inner member 11 may be advantageous for retrieving stones 34 or other calculi deep within the body of the patient. In addition, an inner member 11 having a relatively small diameter may be advantageous for retrieving stones 34 from restricted passageways within the human urinary tract. The inner member 11 may be relatively flexible to facilitate the retrieval of stones 34 located in complex, tortuous body structures. In an exemplary embodiment, the diameter of the inner member 11 may be between about 0.008″ to 0.014″ (about 8 mils to about 14 mils, or about 200 microns to 360 microns).

The sheath 40, the handle 50, and the thumb slide 8 may be formed from any suitable materials used in the art. In some embodiments, the sheath 40, the handle 50, and the thumb slide 8 may be formed from a material discussed above with respect to the elongate region 10. Although FIGS. 1 and 2 illustrate a sheath 40 having a single lumen 30, it is contemplated that the sheath 10 may define more than one lumen 30. The sheath 40 may be dimensioned to fully enclose the inner member 11 when the basket 16 is in the retracted position.

As described earlier, some preferred embodiments of the invention include a one-piece inner member 11 of integrally formed elongate region 10, basket 16, and tip 18. In such embodiments, no mechanical joints are used to attach the basket 16 to the elongate region 10 and/or the tip 18. Elimination of the mechanical joints reduces the overall cross-sectional size of the inner member 11, allowing a reduction in the diameter of the lumen 30. Reduction in the diameter of the lumen 30, in turn, reduces the cross-sectional size of the device 100, thereby reducing patient discomfort. Reduction in size of the device 100 also increases the capability of device 100 to traverse through restricted or tortuous body cavities. All other variables remaining the same, the flexibility (the inverse of flexural rigidity) of the device 100 is inversely proportional to the square of the device diameter. That is, a small decrease in device diameter may significantly increase the device flexibility. Increased flexibility of the device 100 increases the maneuverability of the device 100 within the body. A retrieval device made according to such embodiments can be in the range of approximately 1 Fr. in diameter.

Elimination of mechanical joints in the inner member 11 may also improve the ability of the inner member 11 to slide within the lumen 20 without the joint hindering such movement. Therefore, in embodiments of the invention with one-piece inner member 11, it may be easier to transition the basket 16 from retracted to an expanded configuration and vice versa.

Elimination of the joints may also reduce the possibility of device failure through breakage of a joint. Reduced possibility of inner member 11 breakage may increase the durability of the device 100. In addition, it may be safer to use a device 100 with a one-piece inner member 11 due to the reduced possibility of broken pieces of the inner member 11 being trapped within the body. Elimination of joints may also reduce the possibility of the joint scarring or tearing body tissue during operation.

Fabrication of an inner member 11 with a one piece inner member 11 may also be simpler and faster due to elimination of process steps. In addition to the elimination of one or more joining steps, the creation of a basket 16 on a one-piece inner member may be easier. For instance, for an inner member 11 made of a wire, a distal region can be split into two with one cutting operation to create two legs 20 of the basket 16. The inner member 11 may then be rotated and the two legs 20 split again in one cutting operation to create four legs 20. That is, a total of two cutting operations may create four legs 20. By contrast, for an inner member 11 made of, for example, a tube, two cutting operations may be required to separate one single leg.

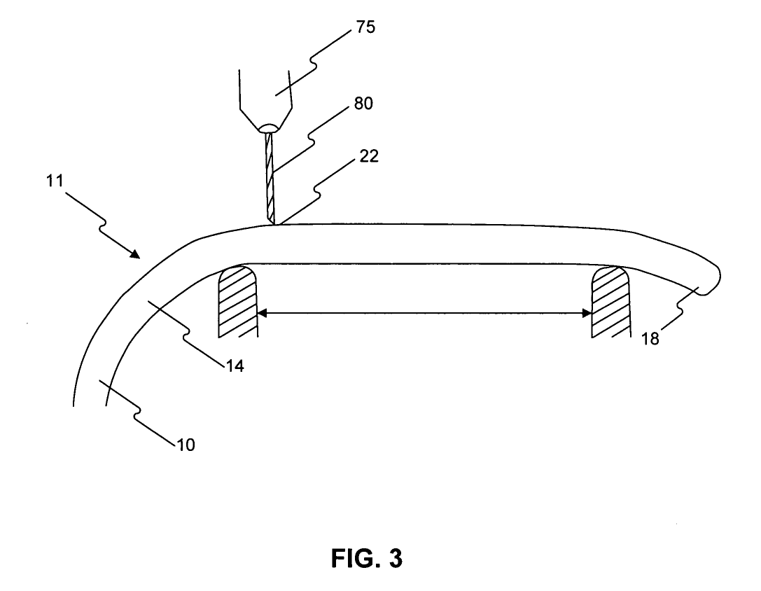

In some embodiments of the current disclosure that achieve at least some of these advantages, the basket 16 may be formed by slicing a wire. The legs 20 of the basket 16 may be formed by slicing a length of material at the distal end of the inner member 11. Any process, such as laser cutting, chemical etching, electrical discharge machining (EDM), machine cutting, etc., capable of slicing a section of the inner member 11 may be used for the slicing operation. FIG. 3 is a schematic illustration of an exemplary laser cutting process used to form the basket 16 at the distal end of the inner member 11. In the embodiment depicted in FIG. 3, the inner member 11 is a nitinol wire. Laser cutting may proceed by focusing a laser beam 80 emanating from a laser on a targeted spot on the distal end of the inner member 11. The spot may be melted or substantially vaporized by the laser beam. The laser beam 80 may burn through the entire thickness of the inner member 11. Water or a flushing fluid may be used to remove debris produced by the laser cutting operation. The laser 75 and/or the inner member 11 may now be moved to burn through an adjacent spot. The laser 75 and/or inner member 11 may be progressively moved to create a longitudinal slot 22 through the inner member 11.

The width of the longitudinal slot 22 may be controlled by controlling the width of the laser or by other means. As explained earlier, one longitudinal slot 22 may create two legs 20 of the basket 16. Multiple longitudinal slots 22 may be cut through the inner member 11 to create more legs 20. For instance, two longitudinal slots 22 ninety degrees (90°) apart may be cut through the entire diameter of the inner member 11 to form four symmetric legs 20. An even number of legs 20 may be formed in this manner. In embodiments, where an odd number of legs 20 are desired, one leg may be further laser cut to form two separate legs 20. A portion of the distal-most end of the inner member 11 may remain uncut to form the tip 18. In some embodiments, the tip 18 may be eliminated and the longitudinal slot 22 may extend to the distal-most end of the inner member 11. In such embodiments, the multiple legs 20 may be connected together only at the member distal end 14.

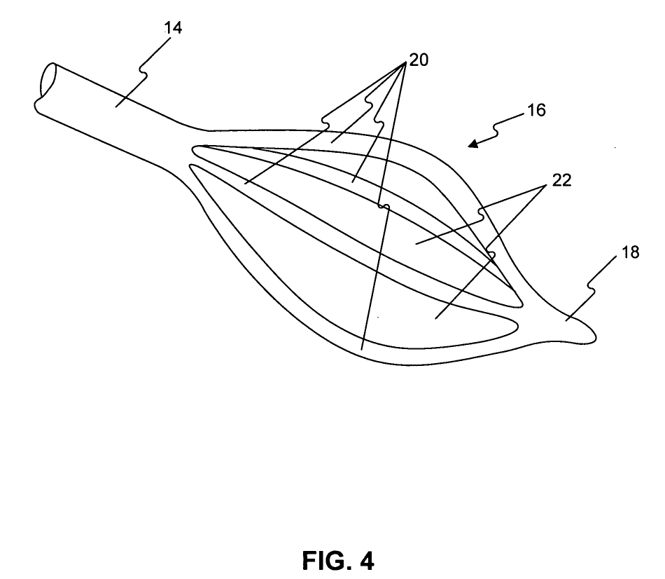

A secondary operation may now separate the legs 20 and increase the space between the legs 20 to form the basket 16. FIG. 4 illustrates the distal end of the inner member 11 with the basket 16. Separating the legs 20 may curve the legs 20 to widen the longitudinal slots 22 created by the slicing operation. The secondary operation may include any automated, semi-automated, or manual operation that is capable of separating the legs 20.

The width and length L of the longitudinal slots 22, and the space between these slots 22 created during the secondary operation, may define the size and mechanical behavior of the basket legs 20. The desired width, length L, and spacing may vary depending on the particular application. For example, for some applications, relatively narrow basket legs 20 separated by large spaces may be desired to retrieve a relatively large stone 34. Each leg 20 may have the same length and width, or the length and width of each leg 20 may vary depending on the particular application. In addition, the width of the longitudinal slots 22 between the legs 20 may vary or be constant.

The shape of the legs 20 may be tailored to suit a particular application. For instance, as depicted in FIGS. 3 and 4, the slicing operation may create substantially straight longitudinal slots 22 resulting in substantially straight legs 20. It is also contemplated that the longitudinal slots 22 may be wave-shaped, v-shaped, saw-shaped, or have any other shape, to create legs 20 with improved stone 34 retrieval characteristics.

The basket 16 may be subjected to additional processing steps to change the physical characteristics and mechanical behavior of the legs 20. For instance, the basket 16 may be heat treated to improve the elastic characteristics of the legs 20. The legs 20 may retain their shape and elasticity after repeated transitions between the expanded and retracted configuration. The shape of the basket 16 may be substantially formed when the basket 16 transitions from the retracted to the expanded configuration. The basket in the expanded configuration may possess a bulbous shape as depicted in FIG. 4. However, it is contemplated that the expanded basket 16 may possess other shapes capable of capturing and/or retrieving a stone 34. The size of the basket 16 may vary depending on the application. In some embodiments, the basket 16 may be sized so as to be capable of capturing a stone 34 in the range of approximately 4 mm to approximately 10 mm along its largest dimension.

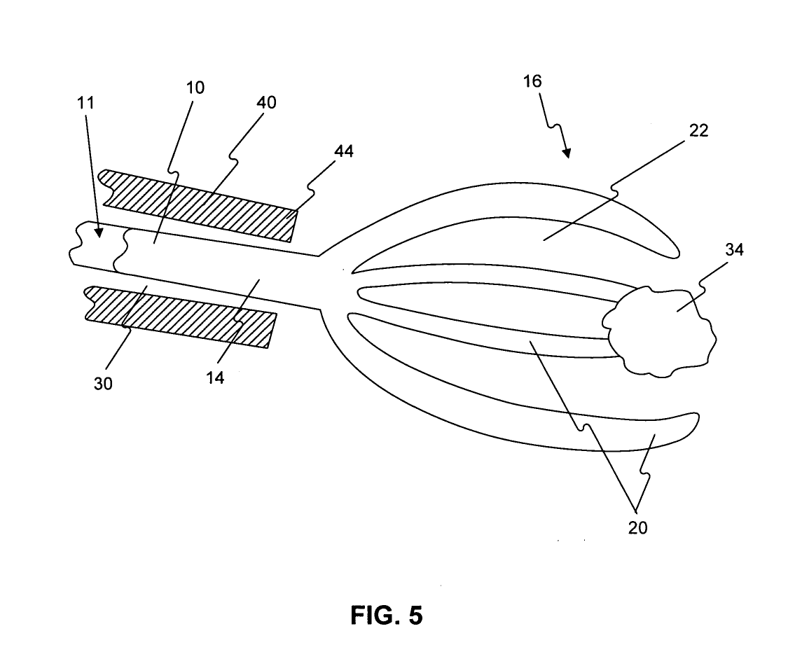

FIG. 5 illustrates another embodiment of the basket 16. In this embodiment, the longitudinal slots 22 extend to the distal end of the inner member 11, eliminating the tip 18. In this embodiment, the legs 20 of the basket 16 may form prongs of a grasping device. In the expanded configuration, the legs 20 of this embodiment may form an open cage. Transitioning the basket 16 from the expanded to the retracted configuration may draw the legs 20 close to each other, thereby grasping a stone 34 between the legs.

In some embodiments, cuts made through the inner member 11 may also form one or more relief features (not shown) at the ends of the longitudinal slots 22. These relief features may include widened ends of the longitudinal slots 22 and may assist in arresting crack propagation that may tend to fracture one or more legs 20. That is, the relief features may assist a longitudinal slot 22 in resisting tearing proximally and/or distally as a result of repeated opening and closing of the basket 16.

In some embodiments, the basket 16 may also include webbing, netting, or any other retrieval material (not shown) disposed between portions of at least two of the legs 20. The retrieval material may be, for example, polyvinylethylene (“PVE”), polyvinyl alcohol (“PVA”), ePTFE, PTFE, foam, rubber, plastic, polyurethane, or any other polymer or composite known in the art. The retrieval material may extend partially or completely along the length of the legs 20 between which it is disposed, and may act as a sieve between those legs 20. The retrieval material may allow, for example, fluid or other material to pass through, while preventing stones 34 from escaping the basket 16. The retrieval material may be attached to the legs 20 by welding, grafting, tying, or any other attachment method known in the art. The legs 20 of the basket 16 may also be coated with a sheet of protection material (not shown) to protect the legs 20 during processes such as, for example, laser lithotripsy.

The configurations, materials, and other characteristics of the basket 16 described herein permit the basket 16 to assume a retracted configuration within the sheath 40 for delivery to a treatment site, and an expanded configuration for use at the treatment site. At least some aspects of the present disclosure may be used, for example, to retrieve a stone 34, calculus, or other material from any location within the body, such as, for example, in the urinary tract of the patient. An exemplary application of the disclosed retrieval device 100 will now be described.

The device 100 may be inserted through the urethra of the patient or, alternatively, the device 100 may be inserted percutaneously to a treatment site. The treatment site within the body may correspond to the location of a targeted stone 34. The stone 34 targeted for retrieval may be a kidney stone, a struvite, an uric acid stone, a cystine stone, or other solid deposit commonly removed from a body structure or passageway within the body. Such stones 34 may contain various combinations of chemicals, including, but not limited to, calcium, oxalate, and phosphate. The stone 34 may be of any size and could have a length or diameter of approximately 1 mm to 12 mm. These lengths and diameters are merely exemplary, and aspects of the present disclosure may assist in the retrieval of stones larger or smaller than those discussed herein. Stones 34 may be of any shape and could be, for example, flat, round, smooth, or jagged. The device 100 may retrieve stones 34 that are both impacted and free floating.

The device 100 may be advanced to the treatment site through an access sheath (not shown), stent, or other access or dilatation device known in the art. In addition, the device 100 may be used in conjunction with an endoscope (not shown) or other type of intracorporeal scope known in the art. The endoscope may advance through the body to the treatment site. Once the treatment site has been reached, the device 100 may be fed through an access port of the endoscope to gain access to the stone 34.

While being advanced to the treatment site, the basket 16 of the device 100 may be in the retracted configuration within the sheath 40. The retracted configuration (shown in FIG. 1) may minimize the size of the diameter of the device 100 and may assist in advancing the device 100 through the endoscope. The sheath distal end 44 may exit the endoscope proximate to the stone 34. Upon exit, the user may transition the basket 16 to its expanded configuration. The basket 16 may then be manipulated relative to the stone 34 so as to capture the stone 34 within the basket 16 (as shown in FIG. 2). In the embodiment of the basket depicted in FIG. 5, capturing the stone 34 in the basket 16 may include positioning the basket close to (or abutting the stone 34), and retracting the inner member 11 into the sleeve 40. Retraction of the inner member 11 forces the legs 20 of the basket 16 together, capturing the stone 34 between them. Once captured, the stone 34 may be retrieved by extracting the device 100 from the body of the patient.

If, however, the targeted stone 34 is too large (in some embodiments, larger than approximately 3 mm) to be safely removed from the body, the user may first reduce the size of the stone 34 prior to removal. In this case, the device 100 may be used to capture the stone 34 as described earlier. The user may then perform a stone-reduction process, such as, for example, laser lithotripsy, to break up or otherwise reduce the size of the stone 34. The device 100 may act as a backstop during such a process and may assist in preventing particles of the stone 34 from migrating or escaping from the treatment site during (or after) the stone-reduction process. In such a process, a laser fiber (not shown) or other conventional device may be used to break up the stone 34. The laser fiber may be delivered to the stone 34 through an access port of the endoscope (or a lumen of the sheath 40) and used to fragment (reduce the size of) the stone 34. Once the stone 34 has been fragmented, the basket 16 may be swept across the area to sweep the stone fragments into the basket 16. The captured stone fragments may then be extracted from the body.

Other embodiments of the invention will be apparent to those skilled in the art from consideration of the specification and practice of the invention disclosed herein. For example, the inner member 11 and/or the sheath 40 may include a plurality of flexibility features configured to increase the flexibility of the device 100. The flexibility features may be formed by removing a portion of the inner member 11 and/or the sheath 40. It is intended that the specification and examples be considered as exemplary only, with the true scope and spirit of the invention being indicated by the following claims.

We claim:

1. A retrieval device, comprising:
a sheath including a lumen extending longitudinally through the sheath;
an elongate member slidably disposed within the lumen; and
a basket at a distal end of the elongate member, the basket including a plurality of legs formed integrally with the elongate member, wherein the elongate member and the basket are formed of a single wire, and the legs are made by splitting a length of the single wire.

2. The retrieval device of claim 1, further comprising a tip at a distal end of the plurality of legs, the tip being formed integrally with the elongate member and the plurality of legs.

3. The retrieval device of claim 2, wherein the elongate member, the basket, and the tip are formed integrally without joints.

4. The retrieval device of claim 1, wherein the plurality of legs are formed by one or more cuts extending all the way through a diameter of the elongate member.

5. The retrieval device of claim 4, wherein one cut forms two legs of the plurality of legs.

6. The retrieval device of claim 4, wherein two cuts form four legs of the plurality of legs.

7. The retrieval device of claim 1, further comprising a handle at a proximal end of the elongate member, wherein the handle is configured to transition the basket from a retracted configuration to an expanded configuration.

8. The retrieval device of claim 7, wherein the retracted configuration is a configuration where the basket is disposed within the lumen, and the expanded configuration is a configuration where the basket is disposed external to the lumen.

9. The retrieval device of claim 7, wherein the basket in the expanded configuration forms a closed cage with the plurality of legs having a bulbous shape.

10. The retrieval device of claim 7, wherein the basket in the expanded configuration forms an open cage with the plurality of legs forming prongs of a grasping device.

11. The retrieval device of claim 1, wherein the elongate member and the plurality of legs are made of nitinol.

12. The medical device of claim 1, wherein a diameter of the elongate member is between about 8 mils and about 14 mils.

13. A method of manufacturing a medical device, comprising:
splitting a length of wire at a distal end of the wire to form a plurality of legs separated by longitudinal slots;
shaping the plurality of legs to define a basket; and
disposing the wire within a longitudinal lumen of a sheath such that the basket is proximate to a first end of the sheath.

14. The method of claim 13, wherein splitting the length of wire includes one of laser cutting, chemical etching, die cutting, and mechanically slicing.

15. The method of claim 13, wherein splitting a length of wire includes cutting all the way through a diameter of a section of the wire to form two legs of the plurality of legs.

16. The method of claim 15, wherein splitting a length of wire further includes rotating the wire and cutting the section of the wire all the way through the diameter to form four legs of the plurality of legs.

17. The method of claim 13, wherein the wire is made of nitinol.

18. The method of claim 13, wherein a diameter of the wire is between about 8 mils and about 14 mils.

19. The method of claim 13, wherein the length of wire is separated from a distal-most end of the wire by a small section of the wire.

20. The method of claim 19, wherein shaping the plurality of legs includes curving the plurality of legs to form a closed basket having a bulbous shape.

21. The method of claim 13, wherein the length of wire abuts a distal-most end of the wire.

22. The method of claim 21, wherein shaping the plurality of legs includes curving the plurality of legs to form an open cage with the plurality of legs forming prongs of a grasping device.

23. The method of claim 13, wherein disposing the wire includes slidably disposing the wire such that the basket is configured to transition from a first configuration to a second configuration, the first configuration being a configuration where the basket is disposed within the lumen and the second configuration being a configuration where the basket is disposed external to the lumen.

24. A method of removing matter from the body of a patient, comprising:
providing a medical device including a sheath defining a lumen, an elongate member slidably disposed within the lumen, and a basket having a plurality of legs, the elongate member and the basket made of a single wire, and the plurality of legs formed by splitting a length of the single wire at a distal end of the single wire;
advancing the medical device to a treatment site within the body of the patient;
capturing the matter within the basket of the medical device; and
removing the medical device from the body of the patient.

25. The method of claim 24, wherein the elongate member and the basket are formed integrally without joints.

26. The method of claim 24, wherein capturing the matter includes transitioning the basket from a retracted configuration to an expanded configuration, the retracted configuration being a configuration where the basket is disposed within the lumen, and the expanded configuration being a configuration where the basket is disposed external to the lumen.

27. The method of claim 26, wherein the length of the single wire is separated from a distal-most end of the single wire by a small section of the single wire, and the basket is configured to have a bulbous shape in the expanded configuration.

28. The method of claim 26, wherein the length of the single wire abuts a distal-most end of the single wire, and the basket is configured to form an open cage with the plurality of legs forming prongs of a grasping device in the expanded configuration.

29. The method of claim 24, further including immobilizing the matter with the plurality of legs of the basket.

30. The method of claim 29, further including fragmenting the matter.

31. The method of claim 30, further including sweeping at least a portion of the matter from the treatment site with the plurality of legs of the basket.

32. The method of claim 24, wherein the matter is one of a kidney stone, a struvite, an uric acid stone, a cystine stone, and a solid deposit.

33. The method of claim 24, wherein one cut forms two legs of the plurality of legs.

34. The method of claim 24, wherein two cuts form four legs of the plurality of legs.
